(19) United States
(12) Patent Application Publication
(10) Pub. No.: US 2026/0265177 A1
(43) Pub. Date: Sep. 10, 2026

(54) BICYCLIC COMPOUNDS AS INACTIVATORS OF OAT AND GABA-AT

(71) Applicant: Northwestern University, Evanston, IL (US)

(72) Inventors: Allison Nicole Devitt, Evanston, IL (US); Richard Bruce Silverman, Evanston, IL (US)

(21) Appl. No.: 19/166,342

(22) PCT Filed: Mar. 18, 2024
(86) PCT No.: PCT/US2024/020388

§ 371 Date: Sep. 17, 2025

Related U.S. Application Data
(60) Provisional application No. 63/490,946, filed on Mar. 17, 2023.

(51) Int. Cl. C07C 229/50; A61K 31/025

(52) U.S. Cl. CPC C07C 229/50; A61K 31/025

(57) ABSTRACT

Disclosed are bicyclic compounds for use as inhibitors of aminotransferases such as gamma-aminobutyric acid amino-transferase (GABA-AT) and/or ornithine aminotransferase (OAT). The disclosed bicyclic compounds and the compositions containing the same may be used for treating diseases and disorders associated with GABA-AT and/or OAT activity, such as neurological and/or cell proliferative diseases or disorders.

### CROSS-REFERENCE TO RELATED APPLICATIONS

The present application claims priority to U.S. Provisional Patent Application No. 63/490,946, filed Mar. 17, 2023, the entire contents of which are hereby incorporated by reference.

### STATEMENT REGARDING FEDERALLY SPONSORED RESEARCH

This invention was made with government support under grant number CA260250 awarded by the National Institutes of Health. The government has certain rights in the invention.

### BACKGROUND OF THE INVENTION

Gamma-Aminobutyric acid (GABA) is the major inhibitory neurotransmitter in the central nervous system. GABA is produced in presynaptic GABAergic neurons from L-glutamate by the enzyme glutamic acid decarboxylase (GAD). GABA is then released by the presynaptic GABAergic neurons into the synapse and transported to glial cells. In glial cells, the enzyme GABA aminotransferase (GABA-AT), a pyridoxal 5′-phosphate (PLP)-dependent enzyme, degrades GABA to succinic semialdehyde (SSA), which is further oxidized to succinate and enters the Krebs cycle. GABA-AT also converts α-ketoglutarate from the Krebs cycle to L-glutamate. Because there is no GAD in glial cells, this newly formed L-glutamate is not converted to GABA. It is instead converted to L-glutamine by glutamine synthetase, which is then released from glial cells into the synapse and transported back to GABAergic neurons to complete the metabolic cycle of L-glutamate.

After GABA is released from presynaptic GABAergic neurons, GABA binds to chloride-selective ion channel receptors including (GABAA and GABAC) and to G-protein coupled receptors that are linked to potassium-selective ion channels including (GABAB). Binding of GABA to these receptors causes the receptors to selectively conduct their respective ions and hyperpolarize the postsynaptic membrane, thereby controlling neuronal activity downwardly. Low levels of GABA are linked to many neurological disorders, including epilepsy, Parkinson's disease, Alzheimer's disease, Huntington's disease, and cocaine addiction.

Another pyridoxal 5′-phosphate (PLP)-dependent enzyme belonging to the same evolutionary subgroup as GABA-AT is the enzyme ornithine aminotransferase (OAT). These two enzymes share a high structural homology and, like all aminotransferases, also have very similar catalytic mechanisms. OAT is expressed in many tissues, including liver, kidney, small intestine, brain, and eye and catalyzes the reversible conversion of ornithine and α-ketoglutarate to L-glutamate semialdehyde which cyclizes to Δ1-pyrroline-5-carboxylate and L-glutamate. L-glutamate is then converted by glutamine synthetase to L-glutamine. Glutamine is the most abundant free amino acid in the body and it is essential for growth of both normal and neoplastic cells. However, tumor cells take up glutamine more efficiently than normal cells, and tumor growth is enhanced by glutamine. With respect to glutamine, cancer cells distinguish themselves from normal cells in that they have an increased requirement for glutamine to support anabolic processes that stimulate proliferation.

Because of the structural similarities between OAT and GABA-AT, it has been shown that some inactivators of GABA-AT also inactivate OAT. As such, developing inactivators that inhibit both GABA-AT and OAT can be useful in the treatment of nervous system disorders and cell proliferative disorders.

### BRIEF SUMMARY OF THE INVENTION

Disclosed are compounds, compositions and related methods of use for the inhibition of ornithine aminotransferase (OAT) and/or GABA aminotransferase (GABA-AT). The disclosed compounds, compositions, and methods can be utilized to treat diseases and disorders associated with ornithine aminotransferase activity and/or GABA-AT activity.

The disclosed compounds may be described as bicyclic compounds. In particular, the disclosed compounds may be described as bicyclo[3.1.0]hexane compounds. The disclosed compounds and compositions thereof may be utilized in methods for modulating ornithine aminotransferase (OAT) activity, including methods for treating diseases or disorders associated with OAT activity or expression such as cell proliferative diseases and disorders. The disclosed compounds and compositions thereof may also be utilized in methods for modulating GABA aminotransferase (GABA-AT) activity, including methods for treating diseases or disorders associated with GABA-AT activity such as neurological diseases and disorders.

The disclosed compounds may be directed to a compound of the following formula or a dissociated form, a non-protonated form, a zwitterion form, or a salt thereof:

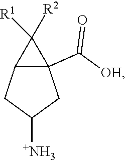

wherein R¹ and R² are independently hydrogen or halo, providing at least one of R¹ and R² is halo.

The disclosed compounds may be directed to an enantiomerically pure compound of the following formula or a dissociated form, a non-protonated form, a zwitterion form, or a salt thereof:

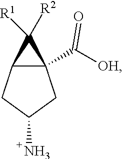

wherein R¹ and R² are independently hydrogen or halo, providing at least one of R¹ and R² is halo.

As indicated, the disclosed compounds may be protonated, for example to form an ammonium substituent, optionally where the compound is present as a salt. The disclosed compounds also may be non-protonated and/or dissociated, for example, where the carboxylic acid substituent is dissociated to form a carboxylate substituent, optionally where the compound is present as a salt. The disclosed compounds may be in zwitterionic form where the compound comprises a protonated ammonium substituent and a dissociated carboxylate substituent, optionally where the compound is present as a salt.

The compounds disclosed herein are without stereochemical or configurational limitation and encompass all stereochemical or configurational isomers, unless stereochemical or configurational limitations are indicated. As illustrated and discussed below, such compounds and/or their intermediates are available as single enantiomers, racemic mixtures from which isomers can be resolved, or diastereomers from which the corresponding enantiomers can be separated. Accordingly, any stereocenter can be(S) or (R) with respect to any other stereocenter(s). As another separate consideration, various compounds can be present as an acid or base salt, either partially or fully protonated, for example at the amino group to form an ammonium substituent, and/or either partially or fully dissociated, for example at the carboxyl group to form a carboxylate substituent. In certain such embodiments, with respect to an ammonium substituent, the counter ion can be a conjugate base of a protic acid. In certain such or other embodiments, with respect to a carboxylate substituent, the counter ion can be an alkaline, alkaline-earth or ammonium cation. Further, it will be understood by those skilled in the art that any one or more the compounds disclosed herein can be provided as part of a pharmaceutical composition comprising a pharmaceutically acceptable carrier component for use in conjunction with a treatment method or medicament.

The disclosed compounds and compositions may be utilized in methods for modulating ornithine aminotransferase (OAT) activity and/or γ-Aminobutyric acid aminotransferase (GABA-AT) activity. Such methods can comprise providing a compound as disclosed herein, or a pharmaceutical composition comprising the compound as disclosed herein, and contacting a medium comprising an OAT and/or a GABA-AT with the compound or the pharmaceutical composition.

In certain embodiments, the disclosed methods may be directed to reducing activity of an OAT expressed by a cancer, which may include but is not limited to hepatocellular cancer (HCC), or other cancers that express or overexpress OAT. Such a method can comprise providing a compound as disclosed herein, or a pharmaceutical composition comprising the compound as disclosed herein, and contacting the cancer cells with the compound or the pharmaceutical composition.

In certain embodiments, the disclosed methods may be directed to treating a cell proliferative disease or disorder in a subject in need thereof. Suitable cell proliferative diseases and disorders may include cancers that express or overexpress OAT such as, but not limited to, hepatocellular carcinoma (HCC). Such a method can comprise administering to such a subject in need thereof a therapeutically effective amount the compound as disclosed herein, or a pharmaceutical composition comprising the compound as disclosed herein.

In certain embodiments, the disclosed methods may be directed to treating a disease or disorder associated with GABA-AT activity in a subject in need thereof. Suitable diseases and disorders may include neurological diseases or disorders that express or overexpress GABA-AT such as, but not limited to, epilepsy. Such a method can comprise administering to such a subject in need thereof a therapeutically effective amount the compound as disclosed herein, or a pharmaceutical composition comprising the compound as disclosed herein.

More generally, the disclosed methods may be directed to inhibiting or inactivating an ornithine aminotransferase and/or γ-aminobutyric acid aminotransferase (GABA-AT). Such a method can comprise providing a compound of the sort discussed above or described below, whether or not part of a pharmaceutical composition, and administering an effective amount of such a compound for contact with an ornithine aminotransferase and/or γ-aminobutyric acid aminotransferase (GABA-AT). Such contact can be, as would be understood in the art, for experimental and/or research purposes or as may be designed to simulate one or more in vivo or physiological conditions. Such compounds can include but are not limited to those illustrated by the following examples, referenced figures, incorporated references and/or accompanying synthetic schemes. In certain such embodiments, such a compound and/or combination thereof can be present in an amount at least partially sufficient to inhibit OAT and/or γ-Aminobutyric acid aminotransferase (GABA-AT), cell proliferation, and/or epilepsy.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows metabolic pathway of GABA.

FIG. 2 shows concentration dependent GABA-AT activity for compound 6.

FIG. 3 shows concentration dependent OAT activity for racemic compound 6.

FIG. 4 shows time-dependent inhibition of GABA-AT by racemic compound 6.

FIG. 5 shows time-dependent inhibition of OAT by racemic compound 6.

FIG. 6 depicts molecular docking of compound 6 in GABA-AT as external aldimine.

FIG. 7 depicts molecular docking of compound 6 in GABA-AT as ketimine.

FIG. 8A depicts molecular docking of compound 6 in hOAT as external aldimine.

FIG. 8B depicts molecular docking of compound 6 in hOAT as the ketimine measuring the distance from lysine to the 8-proton.

FIG. 9 shows time-dependent inhibition of GABA-AT by compound 6.

FIG. 10 shows the time-dependent inactivation of hOAT with compound 6.

FIG. 11 shows the TON of hOAT inactivated by compound 6.

FIG. 12 shows dialysis of hOAT inactivated by compound 6.

FIG. 13 shows the X-ray crystal structure of compound 4.

### DETAILED DESCRIPTION OF THE INVENTION

The disclosed subject matter may be further described using definitions and terminology as follows. The definitions and terminology used herein are for the purpose of describing particular embodiments only, and are not intended to be limiting.

### Chemical Entities

New chemical entities and uses for chemical entities are disclosed herein. The chemical entities may be described using terminology known in the art and further discussed below.

As used herein, the term “halo” refers to a halogen substitution (e.g., —F, —Cl, —Br, or —I).

The term “ammonium” refers to the substituent

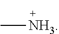

The term “carboxylate” as used herein refers to the substituent —C(O)O⁻.

The term “protic acid” as used herein refers to an acid that is able to release one or more protons and form hydronium ions in an aqueous solution. Examples of protic acid include, but are not limited to, monoprotic acids including hydrochloric acid (HCl), acetic acid (AcOH), nitric acid (HNO₃), benzoic acid (C₆H₅CO₂H), etc., diprotic acids including sulfuric acid (H₂SO₄), carbonic acid (H₂CO₃), hydrogen sulfide (H₂S), etc., triprotic acids including H₃PO₄. The term “conjugate base” refers to the anion that is formed after an acid loses one or more protons. One example of a conjugate base of a protic acid is chloride (Cl⁻).

### Compounds

The disclosed compounds may be directed to a compound of the following formula or a dissociated form, a non-protonated form, a zwitterion form, or a salt thereof:

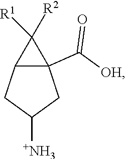

wherein R¹ and R² are independently hydrogen or halo, providing at least one of R¹ and R² is halo.

In some embodiments, the disclosed compounds may be directed to an enantiomerically pure form of the compound of the following formula or a dissociated form, a non-protonated form, a zwitterion form, or a salt thereof:

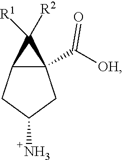

wherein R¹ and R² are independently hydrogen or halo, providing at least one of R¹ and R² is halo.

In some embodiments, at least one of R¹ and R² is F in the compound as disclosed herein. In some embodiments, R¹ and R² are F in the compound as disclosed herein.

In some embodiments, the compound is a salt comprising a substituent that is an ammonium substituent or a carboxylate substituent. In some embodiments, the ammonium substituent has a counter ion that is a conjugate base of a protic acid (e.g. Cl⁻). In some embodiments, the carboxylate substituent has a counter ion that is an alkaline (such as Na⁺ and K⁺), alkaline-earth (such as Mg²⁺ and Ca²⁺) or ammonium cation.

In some embodiments, the compound is in zwitterion form comprising an ammonium moiety and a carboxylate moiety.

In some embodiments, the compound has a formula:

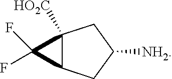

The compounds of the disclosure may be isomeric. In some embodiments, the disclosed compounds may be isomerically pure, wherein the compounds represent greater than about 99% of all compounds within an isomeric mixture of compounds. Also contemplated herein are compositions comprising, consisting essentially of, or consisting of an isomerically pure compound and/or compositions that are isomerically enriched, which compositions may comprise, consist essential of, or consist of at least about 50%, 60%, 70%, 80%, 90%, 95%, 96%, 97%, 98%, 99%, or 100% of a single isomer of a given compound.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” or “+” or “−” depending on the configuration of substituents around the chiral or stereogenic carbon atom and or the optical rotation observed. The disclosed compounds encompasses various stereo isomers and mixtures thereof. Stereoisomers include enantiomers and diastereomers. Mixtures of enantiomers or diastereomers may be designated (±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Also contemplated herein are compositions comprising, consisting essentially of, or consisting of an enantiopure compound and/or compositions that are enantiomer enriched, which compositions may comprise, consist essential of, or consist of at least about 50%, 60%, 70%, 80%, 90%, 95%, 96%, 97%, 98%, 99%, or 100% of a single enantiomer of a given compound (e.g., at least about 95% of an R enantiomer of a given compound).

### Pharmaceutical Compositions

Various non-limiting embodiments of the disclosed compounds and methods of use can be considered with an understanding of a catalytic mechanism of OAT/GABA-AT and mechanism of inactivation of OAT/GABA-AT. In some embodiments, the disclosed subject matter relates to one or more OAT and/or GABA-AT inhibitors, as set forth above, formulated into compositions together with one or more physiologically tolerable or acceptable diluents, carriers, adjuvants or vehicles that are collectively referred to herein as carriers. Compositions suitable for such contact or administration can comprise physiologically acceptable aqueous or nonaqueous solutions, dispersions, suspensions or emulsions, whether or not sterile. The resulting compositions can be, in conjunction with the various methods described herein, for administration or contact with an ornithine aminotransferase. Whether or not in conjunction with a pharmaceutical composition, “contacting” means that an ornithine aminotransferase and one or more inhibitor compounds are brought together for purpose of binding and/or complexing such an inhibitor compound to the enzyme. Amounts of a compound effective to inhibit an ornithine aminotransferase may be determined empirically, and making such determinations is within the skill in the art. Inhibition or otherwise affecting an ornithine aminotransferase activity includes reduction, mitigation and/or modulation, as well as elimination of OAT and/or GABA-AT activity, glutamate production, glutamine synthesis, cell proliferation and/or tumor growth.

In some embodiments, a pharmaceutical composition comprising the compound of as disclosed herein and a pharmaceutically suitable carrier, diluent, or excipient is provided.

The pharmaceutical composition may include the compound in a range of about 0.1 to 2000 mg. In some embodiments, the pharmaceutical composition may include the compound in a range of from about 0.5 to 500 mg. In some embodiments, the pharmaceutical composition may include the compound in a range of from about 1 to 100 mg. The pharmaceutical composition may be administered to provide the compound at a daily dose of about 0.1 to about 1000 mg/kg body weight. In some embodiments, the pharmaceutical composition may be administered to provide the compound at a daily dose of about 0.5 to about 500 mg/kg body weight. In some embodiments, the pharmaceutical composition may be administered to provide the compound at a daily dose of about 50 to about 100 mg/kg body weight. In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 μM, 0.005 μM, 0.01 μM, 0.5 μM, 0.1 μM, 1.0 μM, 10 μM, and 100 μM (e.g., 0.1 μM-1.0 μM).

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are in some embodiments applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use.

Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

Optionally, the disclosed compounds or pharmaceutical compositions comprising the disclosed compounds may be administered with additional therapeutic agents, optionally in combination, in order to treat cell proliferative and/or neurological diseases and disorders. In some embodiments of the disclosed methods, one or more additional therapeutic agents are administered with the disclosed compounds or with pharmaceutical compositions comprising the disclosed compounds, where the additional therapeutic agent is administered prior to, concurrently with, or after administering the disclosed compounds or the pharmaceutical compositions comprising the disclosed compounds. In some embodiments, the disclosed pharmaceutical composition is formulated to comprise the disclosed compounds and further to comprise one or more additional therapeutic agents, for example, one or more additional therapeutic agents for treating cell proliferative and/or neurological diseases and disorders.

It is understood by those skilled in the art that dosage amount will vary with the activity of a particular inhibitor compound, disease state, route of administration, duration of treatment, and like factors well-known in the medical and pharmaceutical arts. In general, a suitable dose will be an amount which is the lowest dose effective to produce a therapeutic or prophylactic effect. If desired, an effective dose of such a compound, pharmaceutically acceptable salt thereof, or related composition may be administered in two or more sub-doses, administered separately over an appropriate period of time.

Methods of preparing pharmaceutical formulations or compositions include the step of bringing an inhibitor compound into association with a carrier and, optionally, one or more additional adjuvants or ingredients. For example, standard pharmaceutical formulation techniques can be employed, such as those described in Remington's Pharmaceutical Sciences, Mack Publishing Company, Easton, PA.

### Methods

Generally, with respect to various embodiments, the disclosed subject matter can be directed to method(s) for the treatment of a pathologic proliferative disorder and/or neurological disorder. As used herein, the term “disorder” refers to a condition in which there is a disturbance of normal functioning. A “disease” is any abnormal condition of the body or mind that causes discomfort, dysfunction, or distress to the person affected or those in contact with the person. Sometimes the term is used broadly to include injuries, disabilities, syndromes, symptoms, deviant behaviors, and atypical variations of structure and function, while in other contexts these may be considered distinguishable categories. It should be noted that the terms “disease”, “disorder”, “condition” and “illness”, are equally used herein.

The terms “treat, treating, treatment” as used herein and in the claims mean ameliorating one or more clinical indicia of disease activity in a subject having a pathologic disorder. “Treatment” refers to therapeutic treatment. Those in need of treatment are mammalian subjects suffering from any pathologic disorder. By “patient” or “subject in need” is meant any mammal for which administration of a compound or any pharmaceutical composition of the sort described herein is desired, in order to prevent, overcome, modulate or slow down such infliction. To provide a “preventive treatment” or “prophylactic treatment” is acting in a protective manner, to defend against or prevent something, especially a condition or disease.

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

As used herein, the term “modulate” means decreasing or inhibiting and/or increasing or augmenting. For example, modulating OAT activity or GABA-AT activity may mean increasing or augmenting OAT activity or GABA-AT, respectively, and/or decreasing or inhibiting OAT activity or GABA-AT, respectively. The compounds disclosed herein may be administered to a subject in need thereof in order to modulate OAT activity and/or GABA-AT activity, for example in order to inhibit OAT activity and/or GABA-AT activity, where the subject has a disease or disorder associated with OAT activity and/or GABA-AT. Ornithine aminotransferase (OAT) activity may be measured using methods disclosed herein and known in the art. (See, e.g., U.S. Published Application Nos. 2018/0098952, 20160128958, and 2012/0245380; and U.S. Pat. Nos. 8,686,041, and 8,211,865; the contents of which are incorporated herein by reference in their entireties). Gamma-aminobutyric acid-aminotransferase (GABA-AT) activity also may be measured using methods disclosed herein and known in the art. (See, e.g., U.S. Published Application Nos. 2019/0315677, 2019/0256489, 2018/0271816, and 2018/0098952; the contents of which are incorporated herein by reference in their entireties).

In some embodiments, the compounds disclosed herein may function as mechanism based enzyme inactivators (MBEI) of OAT and/or GABA-AT. An MBEI is an inert compound that is converted to an active intermediate by an enzyme's normal catalytic machinery. This active intermediate then can form a covalent bond with the enzyme itself or can bind tightly. (See, e.g., Silverman, R. B. “[10] Mechanism-Based Enzyme Inactivators.” Methods in enzymology 1995, 249, 240-283; the content of which is incorporate herein by reference in its entirety). Preferably, the disclosed compounds inactivate OAT and/or GABA-AT with a rate of inactivation (k_{inact}) of greater than about 0.01 min⁻¹, 0.05 min⁻¹, 0.1 min⁻¹, 0.15 min⁻¹, 0.5 min⁻¹. 1.0 min⁻¹, 5.0 min⁻¹, 10 min⁻¹, or higher. Preferably, the disclosed compounds inactivate OAT and/or GABA-AT and have an inhibition constant (K_{I}) of less than about 5 mM, 1 mM, 0.5 mM, 0.1 mM, 0.05 mM, 0.01 mM, 0.005 mM, 0.001 mM, or lower.

According to certain embodiments, the disclosed methods can be specifically applicable for the treatment of malignant proliferative disorders, including malignant proliferative disorders that express ornithine aminotransferase (OAT). As used herein, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. Accordingly, the compounds, compositions, and methods disclosed herein may be used in the treatment of non-solid and solid tumors.

Malignancy, as contemplated herein, may be selected from the group consisting of melanomas, carcinomas, leukemias, lymphomas and sarcomas, which express OAT. Malignancies that can be treated by the methods disclosed herein, including malignancies that express OAT can comprise but are not limited to hematological malignancies (including leukemia, lymphoma and myeloproliferative disorders), hypoplastic and aplastic anemia (both virally induced and idiopathic), myelodysplastic syndromes, all types of paraneoplastic syndromes (both immune mediated and idiopathic) and solid tumors (including bladder, rectum, stomach, cervix, ovarian, renal, lung, liver, breast, colon, prostate, GI tract, pancreas and Karposi). More particularly, according to certain embodiments, the compounds and compositions used in conjunction can be used in methods for the treatment or inhibition of non-solid cancers, e.g. hematopoietic malignancies such as all types of leukemia, e.g. acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma, as well as for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of Vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma, retinoblastoma, carcinoma of the lacrimal gland, sarcoma of the orbit, brain, spinal cord, vascular system, hemangiosarcoma and Kaposi's sarcoma.

In some embodiments, the disclosed compounds and pharmaceutical compositions may be utilized for treating a neurological or psychological disease or disorder in a subject in need thereof. In these methods, the subject may be administered an amount of the compound sufficient to modulate GABA-AT activity. In these methods, the disease or disorder may be a neurological disorder optionally selected from the group consisting of, but not limited to, epilepsy, partial seizures, complex partial seizures, secondary generalized seizures, tonic-clonic seizures, succinic semialdehyde dehydrogenase deficiency (SSADHD), infantile spasms in West's syndrome, Lennox-Gastaut syndrome, tubulous sclerosis, Tourette's syndrome, movement disorders, fibromyalgia, neuropathic pain, migraine related to epilepsy, restless leg syndrome and post-traumatic stress disorder, addiction, obesity, obsessive-compulsive disorders and Alzheimer's disease, and combinations thereof. In these methods, the disease or disorder may be a psychological disorder optionally selected from, but not limited to, the group consisting of general anxiety disorder, pathological or compulsive gambling disorder, compulsive eating, body dysmorphic disorder, hypochondriasis, pathologic grooming conditions, kleptomania, pyromania, attention deficit hyperactivity disorder and impulse control disorders and combinations thereof.

The compounds and compositions disclosed herein may be administered in methods of treatment as known in the art. Accordingly, various such compounds and compositions can be administered in conjunction with such a method in any suitable way. For example, administration may comprise oral, intravenous, intraarterial, intramuscular, subcutaneous, intraperitoneal, parenteral, transdermal, intravaginal, intranasal, mucosal, sublingual, topical, rectal or subcutaneous administration, or any combination thereof.

More generally, the disclosed methods may be directed to affecting, modulate, reducing, inhibiting and/or preventing the initiation, progression and/or metastasis (e.g., from the liver elsewhere or to the liver from any other organ or tissue) of a malignant pathologic proliferative disorder associated with OAT activity. (See, e.g., Lucero O M, Dawson D W, Moon R T, et al. A re-evaluation of the “oncogenic” nature of Wnt/beta-catenin signaling in melanoma and other cancers. Curr Oncol Rep 2010, 12, 314-318; Liu Wei; Le Anne; Hancock Chad; Lane Andrew N; Dang Chi V; Fan Teresa W-M; Phang James M. Reprogramming of proline and glutamine metabolism contributes to the proliferative and metabolic responses regulated by oncogenic transcription factor c-MYC. Proc. Natl. Acad. Sci. USA 2012, 109 (23), 8983-8988; and Tong, Xuemei; Zhao, Fangping; Thompson, Craig B. The molecular determinants of de novo nucleotide biosynthesis in cancer cells. Curr. Opin. Genet. Devel. 2009, 19 (1), 32-37.) In some embodiments, the disclosed methods may be directed to affecting, modulate, reducing, inhibiting and/or preventing the initiation and/or progression of a neurological disease or disorder associated with GABA-AT activity.

As used herein, a “subject” may be interchangeable with “patient” or “individual” and means an animal, which may be a human or non-human animal, in need of treatment. According to some embodiments, the treated subject may be a mammalian subject. Although the methods disclosed herein are particularly intended for the treatment of proliferative and/or neurological disorders in humans, other mammals are included. By way of non-limiting examples, mammalian subjects include monkeys, equines, cattle, canines, felines, mice, rats and pigs.

As used herein, the term “a subject in need thereof” refers to a human or non-human subject that can be treated with any of the compounds or pharmaceutical compositions disclosed herein when the compounds or pharmaceutical compositions are utilized as therapeutic agents. A subject in need thereof may include a subject having a disease or disorder that is associated with a biological activity of ornithine aminotransferase (OAT). In some embodiments of the disclosed subject matter, a subject in need thereof may include a subject having a disease or disorder that is associated with increased expression of OAT (e.g., a subject having a cancer which overexpresses OAT). A subject in need thereof may include a subject having a disease or disorder that is associated with a biological activity of gamma aminobutyric acid aminotransferase (GABA-AT) and/or concentrations of GABA in the subject.

A subject in need thereof may include a subject having a disease or disorder that is associated with ornithine aminotransferase (OAT), for example a disease or disorder that is associated with expression of OAT (e.g., increased expression of OAT) or a disease or disorder that is associated with a biological activity of OAT (e.g., increased activity of OAT for catalyzing synthesis of glutamate and/or glutamine). Diseases and disorders associated with expression of OAT and diseases or disorders that are associated with a biological activity of OAT are known and may include, but are not limited to cell proliferative diseases or disorders such as cancers.

In some embodiments, a subject in need thereof may include a subject having cancer. A subject in need thereof may include a subject having a cancer selected from, but not limited to, liver cancer (e.g., hepatocellular carcinoma (HCC)), and cancers such as multiple myeloma, leukemia, non-small cell lung cancer, colon cancer, cancer of the central nervous system, melanoma, ovarian cancer, renal cancer, prostate cancer, breast cancer, and pancreatic cancer.

A subject in need thereof may include a subject having a disease or disorder that is associated with gamma aminobutyric acid-amino transferase (GABA-AT) or with concentrations of GABA in the subject. A subject in need thereof may include a subject having a disease or disorder that may be treated by increasing the concentration of gamma aminobutyric acid (GABA) in the subject, for example, via administering to the subject a therapeutic agent that inhibits the activity of GABA-AT, thereby increasing the concentration of GABA in the subject. Diseases and disorders associated with a biological activity of GABA-AT and/or concentrations of GABA in a subject are known and may include, but are not limited to neurological diseases and disorders.

In some embodiments, a subject in need thereof may include a subject having a neurological disease or disorder. In some embodiments, a subject in need thereof is a subject having a disease or disorder selected from epilepsy, Huntington's disease, Alzheimer's disease, and Parkinson's disease.

As used herein, a “therapeutic agent” may refer to any agent that is administering to a subject in thereof in order to treat the subject. A therapeutic agent may refer to an agent that modulates the biological activity of OAT, for example where the agent inhibits the biological activity of OAT to catalyze the synthesis of glutamate or glutamine. A therapeutic agent may refer to an agent that modulates the biological activity of GABA-AT, for example where the agent inhibits the biological activity of GABA-AT to degrade GABA to succinic semialdehyde (SSA). Therapeutic agents may include, but are not limited to, small molecules or compounds as disclosed herein. Therapeutic agents may include, but are not limited to, pharmaceutical compositions comprising small molecules or compounds as disclosed herein.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### ILLUSTRATIVE EMBODIMENTS

The following embodiments are illustrative and should not be interpreted to limit the scope of the claimed subject matter.

Embodiment 1. A compound of the following formula or a dissociated form, a non-protonated form, a zwitterion form, or a salt thereof:

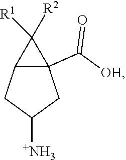

wherein R¹ and R² are independently hydrogen or halo, providing at least one of R¹ and R² is halo.

Embodiment 2. An enantiomerically pure form of the compound of embodiment 1 having the following formula:

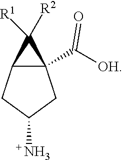

Embodiment 3. The compound of embodiment 1 or 2, wherein at least one of R¹ and R² is F.

Embodiment 4. The compound of any one of embodiments 1-3, wherein R¹ and R² are F.

Embodiment 5. The compound of any one of embodiments 1-4, wherein the compound is a salt comprising a substituent that is an ammonium substituent or a carboxylate substituent.

Embodiment 6. The compound of embodiment 5, wherein the ammonium substituent has a counter ion that is a conjugate base of a protic acid.

Embodiment 7. The compound of any one of embodiments 1-5 in zwitterion form comprising the ammonium substituent and the carboxylate substituent.

Embodiment 8. The compound of any one of embodiments 1-7 of a formula:

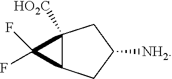

Embodiment 9. A pharmaceutical composition comprising the compound of any one of embodiments 1-8 and a pharmaceutically suitable carrier, diluent, or excipient.

Embodiment 10. A method of modulating ornithine aminotransferase (OAT) activity, the method comprising contacting the compound of any one of embodiments 1-8, or the pharmaceutical composition of embodiment 9, with a medium comprising an OAT, wherein the compound is present in an amount sufficient to modulate OAT activity.

Embodiment 11. The method of embodiment 10, wherein the contact is in vivo.

Embodiment 12. The method of embodiment 15 or 16, wherein the OAT is human ornithine aminotransferase (hOAT).

Embodiment 13. A method of reducing activity of an OAT expressed by a human cancer, the method comprising contacting the compound of any one of embodiments 1-8, or the pharmaceutical composition of embodiment 9, with the cancer expressing an OAT, wherein the compound is present in an amount that is effective to reduce OAT activity.

Embodiment 14. The method of embodiment 13, wherein the contact is in vivo.

Embodiment 15. The method of embodiment 13 or 14, wherein the contact is with a human subject in need of a treatment for cancer.

Embodiment 16. The method of any one of embodiments 13-15, wherein the OAT is human ornithine aminotransferase (hOAT).

Embodiment 17 A method for treating cancer in a subject in need thereof, the method comprising administering to the subject a therapeutically effective amount of the compound of any one of embodiments 1-8, or the pharmaceutical composition of embodiment 9.

Embodiment 18. The method of embodiment 17, wherein the cancer is characterized by expression or overexpression of human ornithine aminotransferase (hOAT).

Embodiment 19. The method of embodiment 17 or 18, wherein the cancer is hepatocellular carcinoma (HCC).

Embodiment 20. A method of modulating γ-Aminobutyric acid aminotransferase (GABA-AT) activity, the method comprising contacting the compound of any one of embodiments 1-8, or the pharmaceutical composition of embodiment 9, with a medium comprising a GABA-AT, wherein the compound is present in an amount sufficient to modulate GABA-AT activity.

Embodiment 21. The method of embodiment 20, wherein the contact is in vivo.

Embodiment 22. A method for treating a disease or disorder associated with GABA-AT activity in a subject in need thereof, the method comprising administering to the subject a therapeutically effective amount of the compound of any one of embodiments 1-8, or the pharmaceutical composition of embodiment 9.

Embodiment 23. The method of embodiment 22, wherein the disease or disorder is a neurological disease or disorder.

Embodiment 24. The method of embodiment 22 or 23, wherein the disease or disorder is epilepsy.

### EXAMPLES

The following examples are illustrative and should not be interpreted to limit the scope of the claimed subject matter.

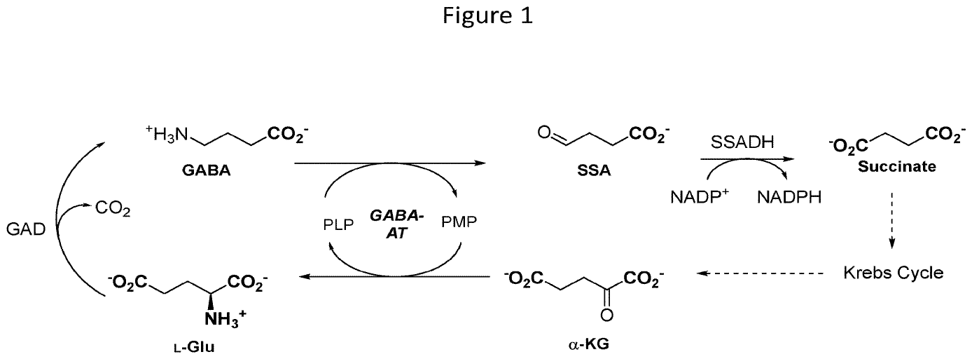

γ-Aminobutyric acid aminotransferase (GABA-AT) is a pyridoxal 5′-phosphate (PLP)-dependent enzyme that catalyzes the degradation of the inhibitory neurotransmitter GABA to succinic semialdehyde, while converting alpha-ketoglutarate to L-glutamate. In addition, the PLP-dependent enzyme L-glutamic acid decarboxylase (GAD) can also catalyze the conversion of L-glutamate to GABA (FIG. 1).1 GABA-AT and GAD regulate the amount of GABA-AT in the brain and are critical for normal function. GABA and L-glutamate serve opposite functions, with GABA acting as a key inhibitory neurotransmitter in mammals while L-glu is excitatory.² GABA is found in many organs including the kidney and pancreas, as well as in glial cells and presynaptic neurons.³ When the concentration of GABA falls below a threshold in the brain, it results in convulsions (seizures) which can be terminated by raising GABA levels.⁴ Epilepsy is broadly defined as any disease characterized by reoccurring seizures, and over one percent of the world can be classified as suffering from epilepsy.⁵ Development of effective anticonvulsants without substantial side effects have been a medicinal focus for centuries. Diphenylhydantoin (Dilantin) is one of the most commonly used anticonvulsants, despite being introduced over 65 years ago and despite not being generally applicable.⁶ Currently, more than 30% of epileptic patients do not respond to any available anticonvulsant drugs.⁷ Inhibition of GABA-AT, which is a major component in the catabolism of GABA, has shown efficacy in dampening excessive neuronal activity without affecting basal neuronal firing levels.⁸ Decreased concentrations of GABA is implicated in several other neurological diseases besides epilepsy, including Huntington's chorea,⁹ Parkinson's disease,¹⁰ and Alzheimer's disease.¹¹ GABA is not suitable to be dosed peripherally as it will not cross the blood brain barrier under normal conditions¹² and is readily effluxes out of the brain¹³.

Mechanism-based inactivators (MBIs) are unreactive small molecules structurally related to the endogenous substrate of the target enzyme.¹⁴ They initially engage the enzyme as a substrate, undergoing modifications by the protein that then create a reactive species that can cause irreversible inhibition of the target enzyme. This can reduce off-target effects and toxicity, as the MBI is not reactive until engaged in the enzyme pocket. Many MBIs operate by forming covalent bonds to the protein itself, but they can also covalently modify the cofactor or form slow-tight binding adducts that functionally inactivate the protein.

Ornithine aminotransferase (OAT) is a related aminotransferase in the same evolutionary subgroup as GABA-AT.¹⁵ OAT is expressed in the mitochondrial matrix of mammal tissues as well as in the liver.¹⁶ Despite limited sequence homology (17%), OAT and GABA-AT share similar active sites with few differences.¹⁷ Specifically, they share key residues including a lysine, aspartate, arginine, and glycine.¹⁸ The lysine forms a covalent tether for PLP with an aldimine linkage, and acts as the base to mediate the isomerization of the substrate to a ketamine. The aspartate forms a key hydrogen bonding residue with the pyridine ring of PLP, and the arginine residue provides a key interaction with the carboxyl group of the substrate anchoring the substrate and facilitating catalytic turnover. The main differences between the active sites of GABA-AT and OAT are the presence of two tyrosine's in OAT that are replaced with Phe351 and Ile72 in GABA-AT¹⁸.

MBIs of aminotransferases undergo a shared initial condensation reaction onto the PLP cofactor of the enzyme. After condensation onto PLP, which forms an external aldimine, a base-mediated isomerization can form a ketamine intermediate which can then undergo a number of different steps.²³ As shown in Scheme 1, compound 6 can act as a mechanism-based inactivator of ornithine aminotransferase (OAT) due to the more polar active site of OAT stabilizing the ketamine intermediate. Compound 6 may undergo a second deprotonation of a gamma-proton that would ring-open the difluoro cyclopentane to either eliminate fluoride (S3) or form the enolate of the carboxylate (S2). Enolate S2 may reform the carbonyl and eliminate fluoride to converge on intermediate S3. A rapid tautomerization to the enamine of S3 would aromatize the complex and provide a slow-tight binding complex that would act as a covalent bond to the cofactor thereby inactivating the enzyme. The same inactivation mechanism may occur in GABA-AT similar to the above mechanism in OAT. S5 may be the turnover product, resulting from hydrolysis of the inhibitor from the enzyme prior to the second deprotonation step.

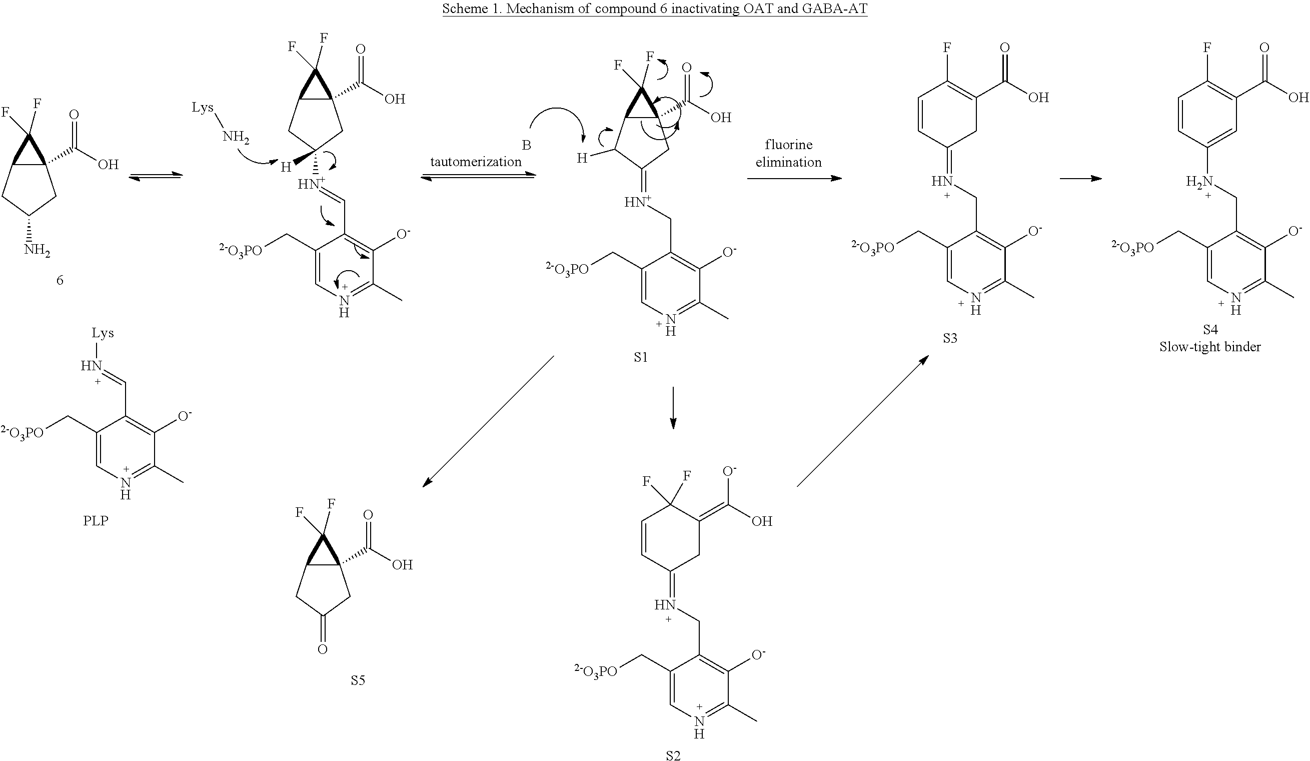

### Modeling

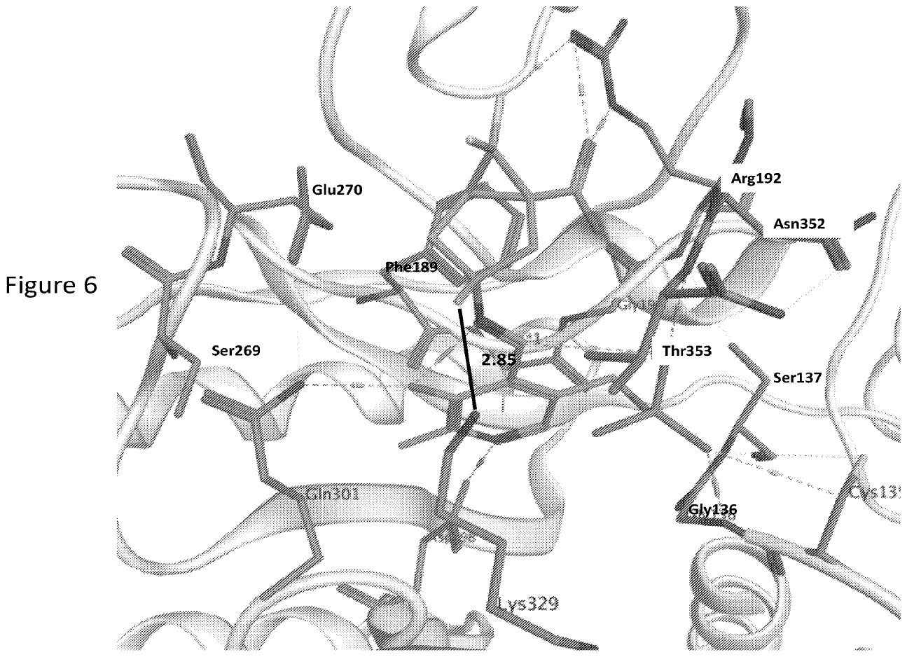

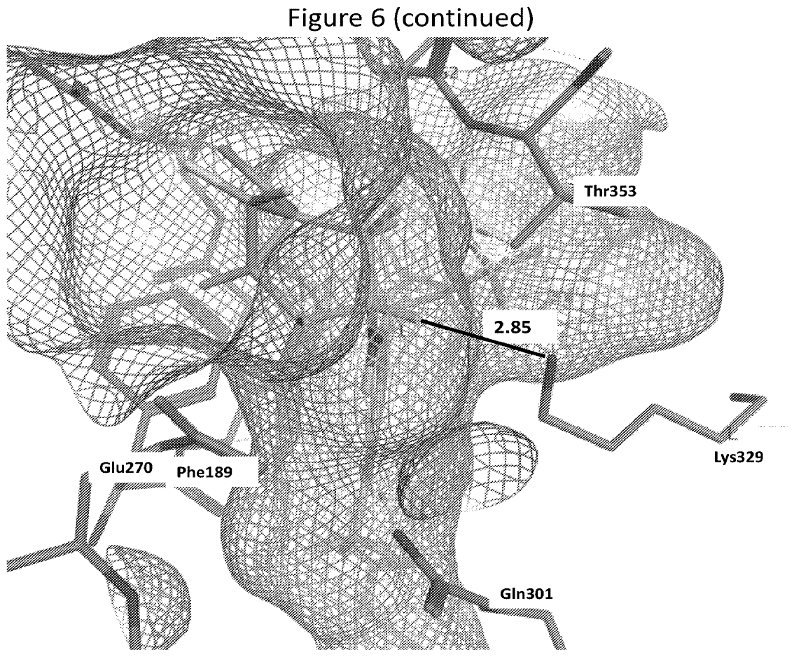

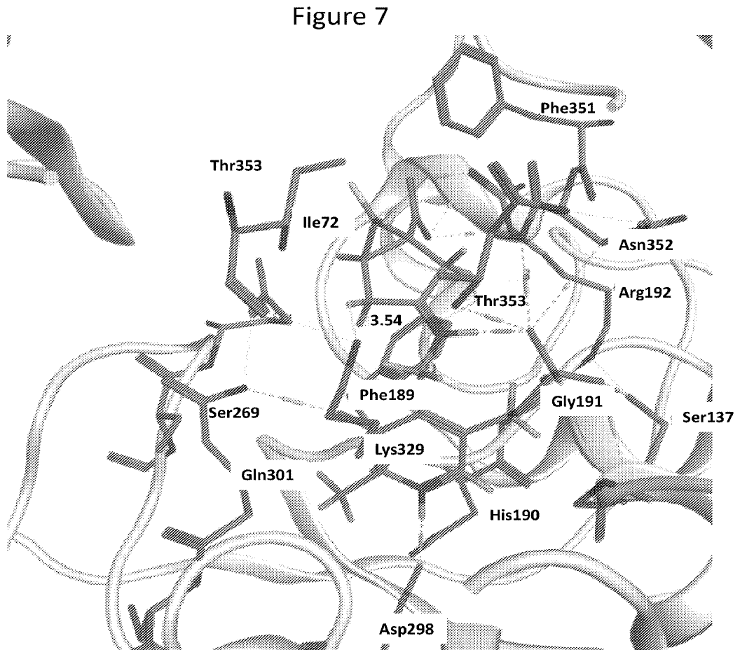

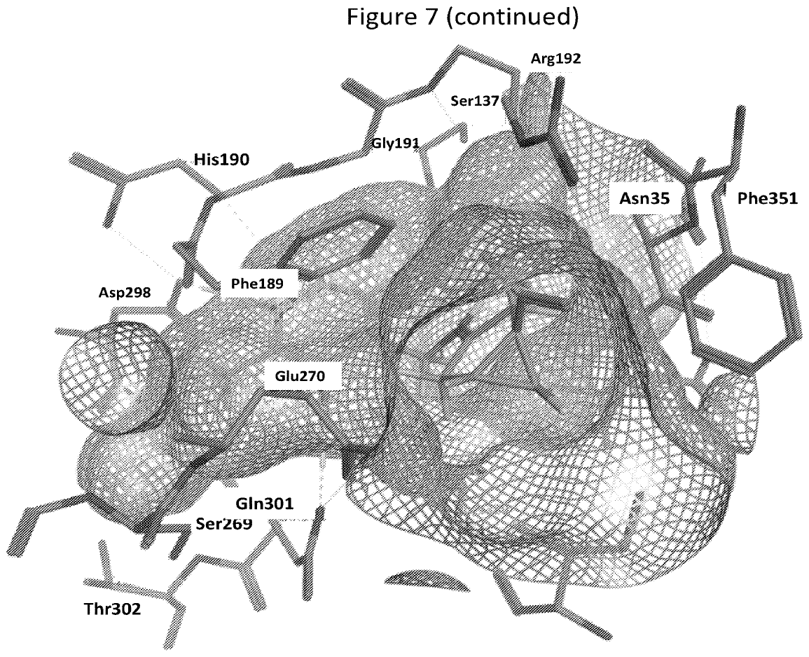

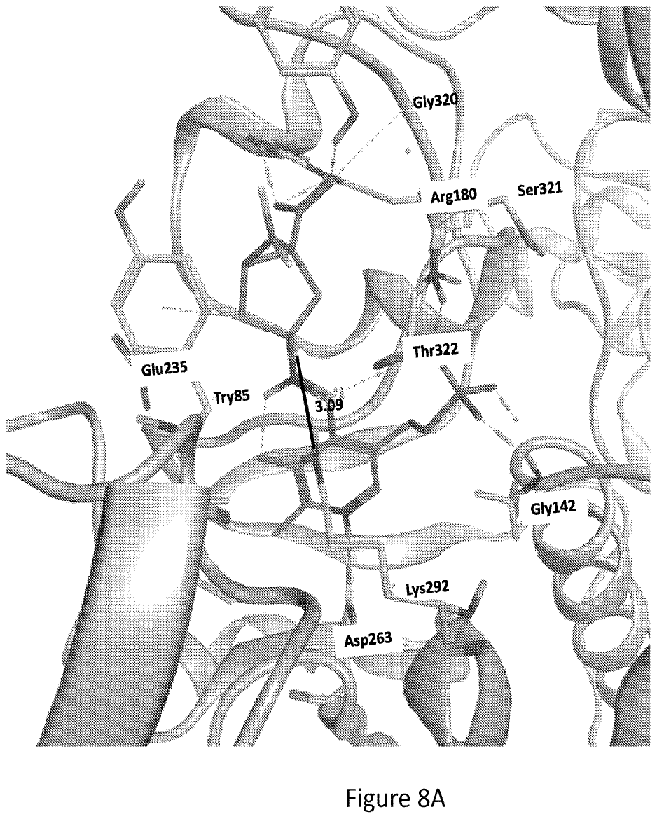

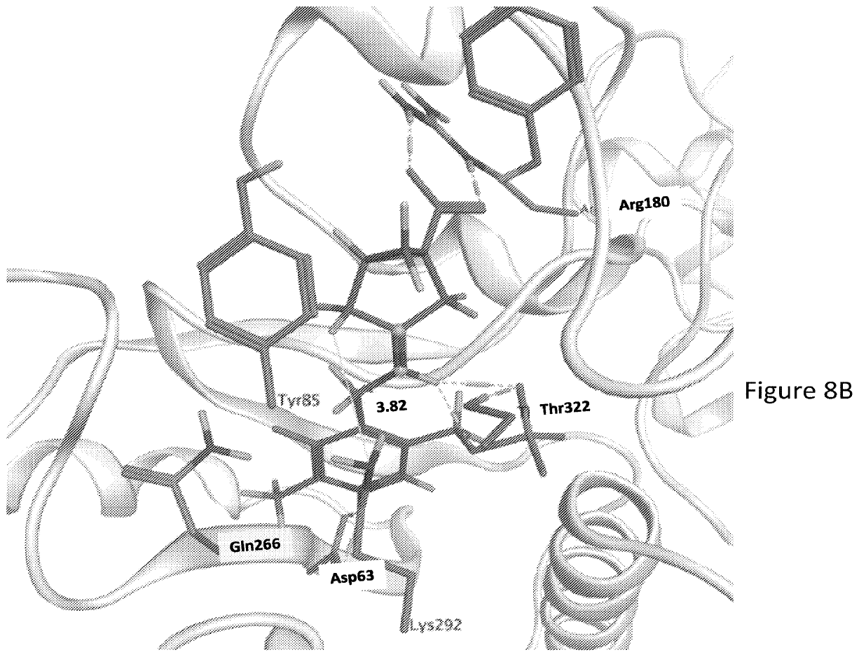

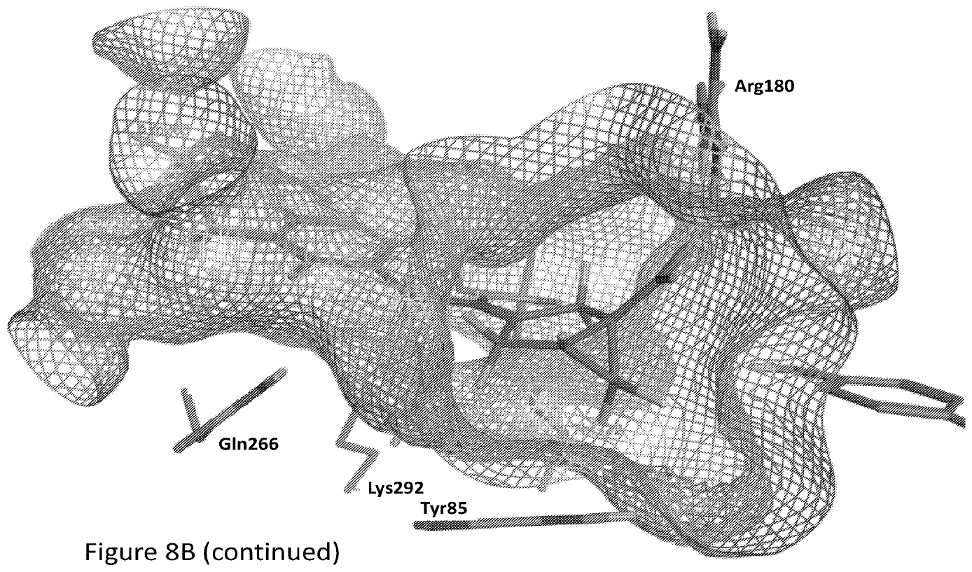

To determine if the formation of both the aldimine and ketamine of 6 complexed to PLP was possible, modeling was employed to compare the binding of both intermediates in GABA-AT and OAT (FIGS. 6-8). The catalytic lysine residue can be in close proximity to the acidic proton for the formation of the ketamine and for the second deprotonation event in GABA-AT (FIG. 7). Modeling the surface of the active site also shows that the difluorocyclopropane, which could have been too large to fit into the GABA-AT active site, is well tolerated and is occupying part of the slightly hydrophobic channel of the GABA-AT active site. In comparison, the distance between the relevant protons and the catalytic lysine is further away for both the ketamine and aldimine in OAT (FIG. 8).

### Biological Assay Results

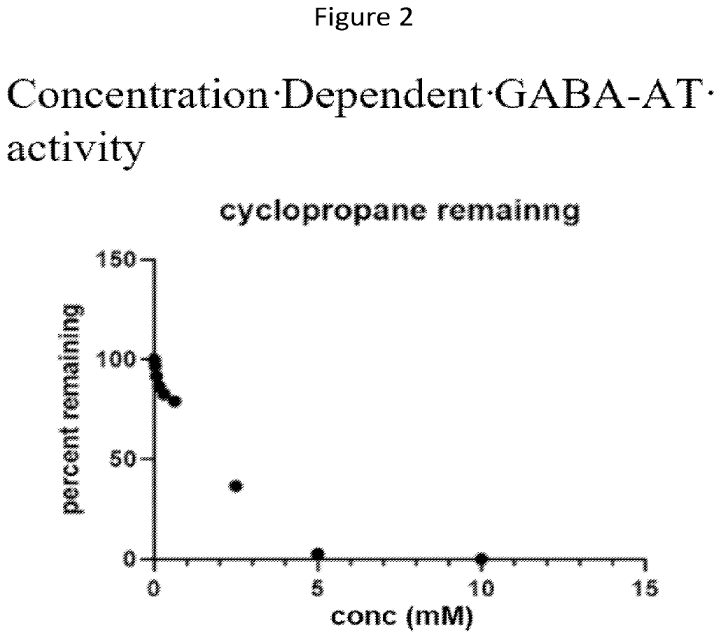

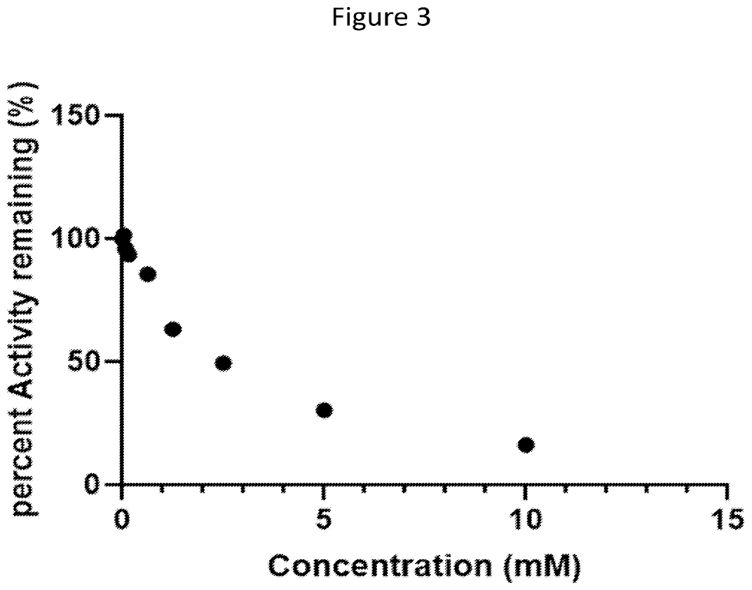

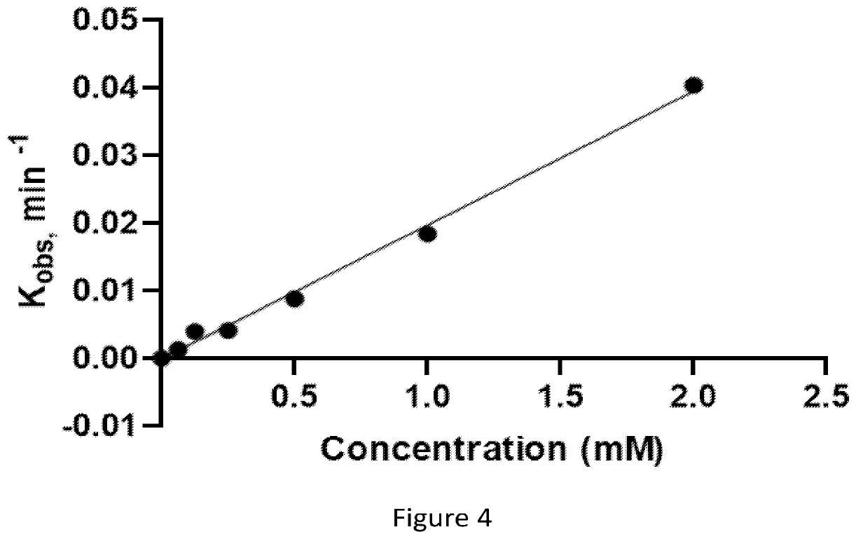

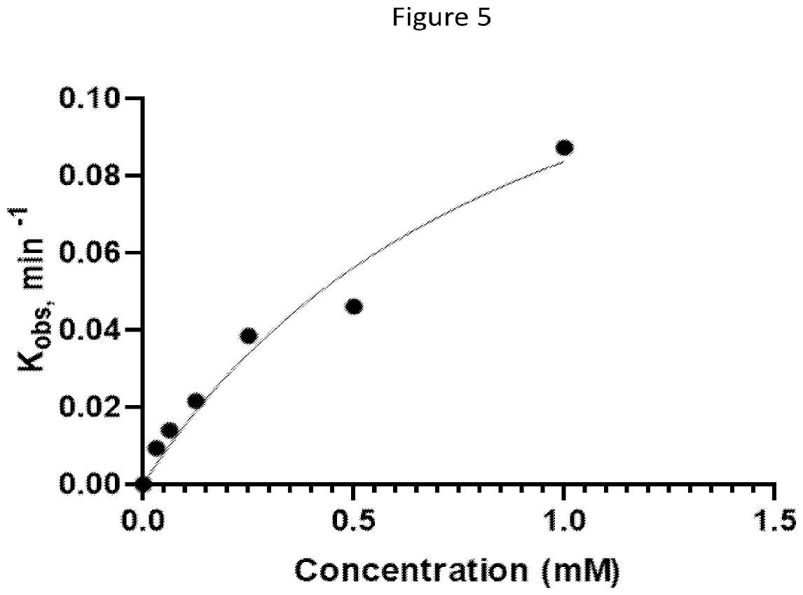

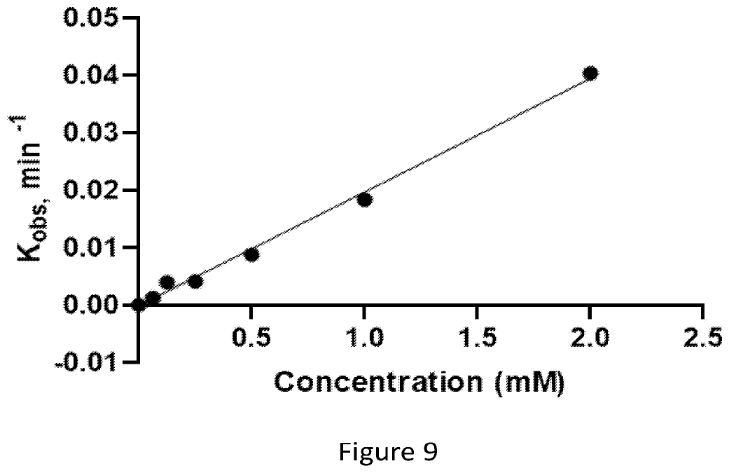

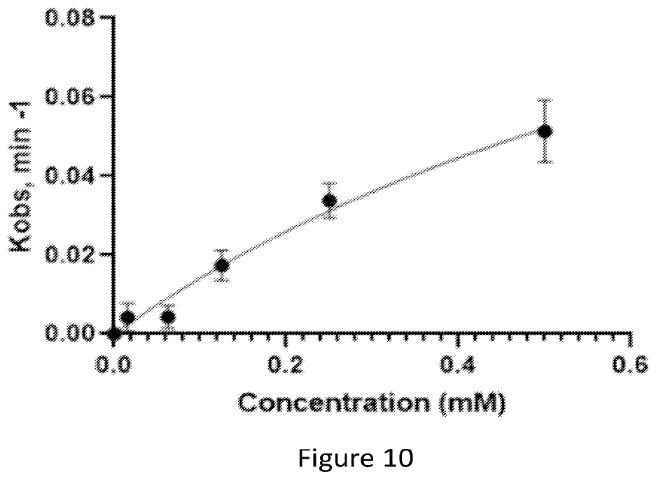

In concentration dependent assays against both OAT and GABA-AT, the racemic version of 6 fully inhibited GABA-AT at 5 mM (FIG. 2) and OAT showing 30% remaining activity at 10 mM of 6 (FIG. 3). Time-dependent inhibition of both GABA-AT and OAT were explored with racemic 6 (FIGS. 4 and 5). Time-dependent inhibition of OAT showed a k_{inact}/Kᵢ of 0.855 1/mM min (FIG. 5), but the time-dependent inactivation was weak in GABA-AT (FIG. 9). Time dependent inactivation for GABA-AT at 0.15 mM is shown in FIG. 4. Racemic Vince lactam was used for the aforementioned biological assays.

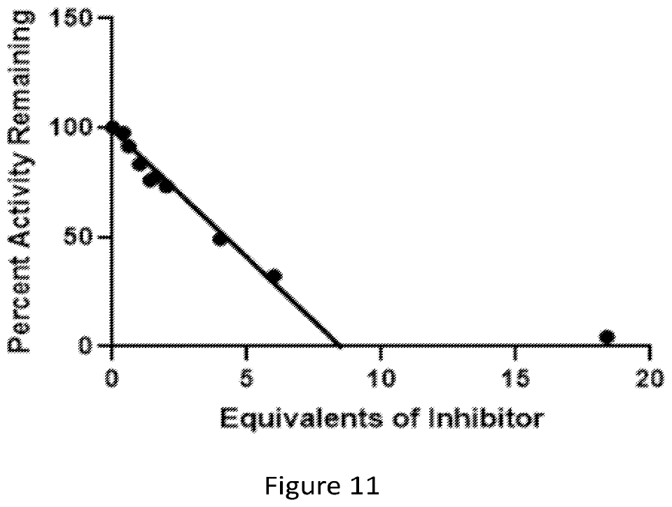

The biochemical assays of enantiopure 6 found that the k_{inact}/K_{I} of 6 in hOAT was 0.156 1/mM min (FIG. 11).

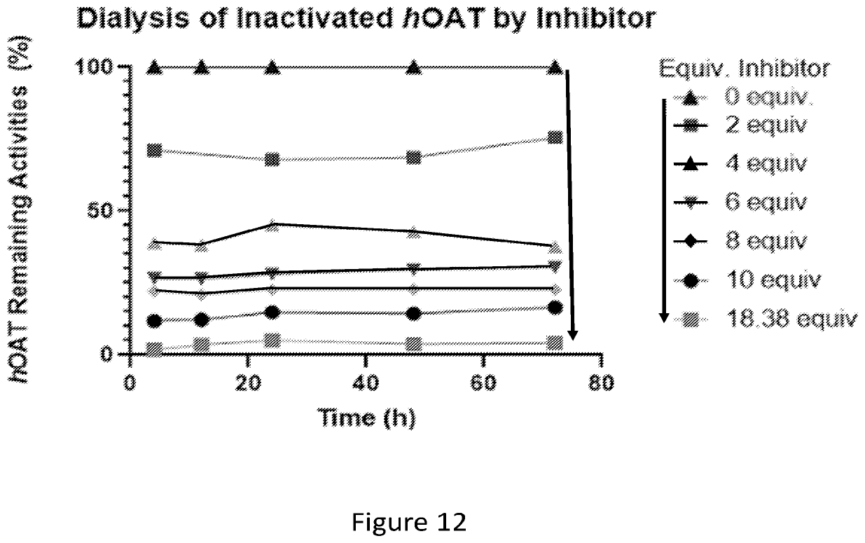

The turnover number and partition ratio of 6 were determined by titrating a known amount of hOAT with varying equivalents of inactivator (FIG. 12). The linear relationship of the remaining enzymatic activity was extrapolated to yield the number of equivalents of 6 required to inactivate the enzyme completely. From this we determined the turnover number of 6 to be 8.5 equivalent and the partition ratio to be 7.5. Based on our understanding of the mechanism, we believe that the turnover number may indicate that the second deprotonation is slow, which would allow the ketimine time to hydrolyze in a higher percentage than if the second deprotonation occurred rapidly.

Samples of hOAT that had been inactivated by varying equivalents of 6 were dialyzed at 5° C. for 72 hours. At 4-, 12-, 24-, 48-, and 72-hour time points, aliquots were taken and the activity of the hOAT was measured. No enzymatic activity was recovered after 72 hours of dialysis for hOAT inhibited by 6 (FIG. 12), indicating complete irreversible inhibition of hOAT by 6.

### Synthesis Example 1

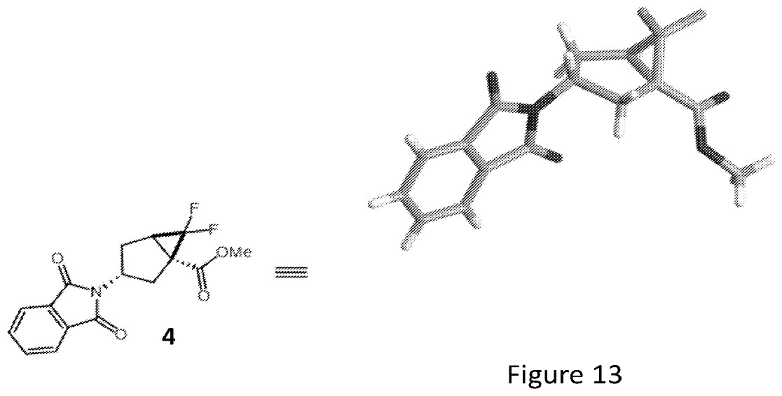

The synthesis of 6 is shown in Scheme 2. The synthesis begins with enantiopure Vince lactam (1) which was ring opened to a free amine with methanolic HCl, Boc-protected, followed by isomerization of the alkene to the more thermodynamically favored unsaturated ester 2. The Boc group can then be exchanged to a phthalimide via deprotection with TFA and protection of the resulting free amine with phthalic anhydride. The resulting phthalimide 3 can then be subjected to TEDA with catalytic NaF at reflux to yield the desired difluorocyclopropane 4 as a single regioisomer. We confirmed the stereochemistry of 4 with 2-D NMR spectroscopy as well as through obtaining a single crystal X-ray structure (FIG. 13). Deprotection of the phthalimide with hydrazine provides the free amine 5, which can then be further deprotected with aqueous HCl to the HCl salt of 6.

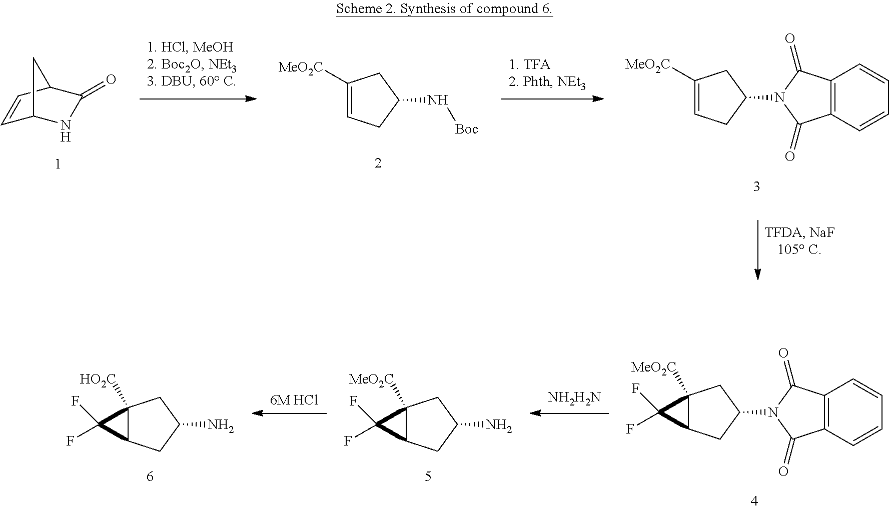

### Methyl(R)-4-((tert-butoxycarbonyl)amino)cyclopent-1-ene-1-carboxylate (2)

(S,4R)-2-azabicyclo[2.2.1]hept-5-en-3-one (1) (20.0 g, 183 mmol, 1.0 equiv) was dissolved in HCl in MeOH (400 mL, 3M), and the resulting solution was heated to 60° C. for 1 hour or until the starting material was no longer observed. The solution was cooled then concentrated to a white solid, which was used crude in the next reaction.

The resulting solid was dissolved in DCM (300 mL) and triethylamine (32.65 mL, 1.3 equiv), after which Boc anhydride (99.7 g, 457 mmol, 2.5 equiv) was added portion wise to the solution. The reaction was allowed to stir at room temperature overnight under a blanket of nitrogen. Upon completion of the reaction, the solution was washed with 20% citric acid (2×70 mL), brine (100 mL), and dried over Na₂SO₄. The solution was concentrated down to a white solid and used crude in the next reaction.

The crude white solid was then dissolved in anhydrous THF (300 mL) and DBU (38 mL, 248 mmol, 1.4 equiv) was added to the reaction mixture. The reaction was heated to 70° C. for 5 hours. The reaction was cooled, diluted with DCM (400 mL), washed with 15% citric acid (50 mL), and brine (50 mL), and dried over Na₂SO₄. The organic layer was concentrated, and the resulting residue was purified by silica gel chromatography (0-20% EtOAc in hexane) to afford 2 as a white solid (37.4 g, 85%). ¹H NMR (500 MHz, CDCl₃) δ 6.71 (p, J=2.2 Hz, 1H), 4.36 (s, 1H), 3.74 (s, 3H), 3.01-2.86 (m, 2H), 2.41 (dddt, J=30.1, 18.8, 4.5, 2.2 Hz, 2H), 1.44 (s, 9H). ¹³C NMR (126 MHz, CDCl₃) δ 165.10, 155.30, 141.18, 134.42, 79.46, 51.55, 50.15, 41.16, 39.14, 28.39. HR-MS (ESI) calculated [M+Na]⁺for C₁₂H₁₉NO₄⁺=264.1212, found: 264.1036.

### Methyl(R)-4-(1,3-dioxoisoindolin-2-yl)cyclopent-1-ene-1-carboxylate (3)

To 2 (7.5 g, 311 mmol, 1.0 equiv) was added DCM (600 mL) and TFA (119 mL, 1.55 mol, 50.0 equiv), and the reaction mixture was stirred at room temperature overnight. Upon completion, the reaction was concentrated and azeotroped with anhydrous toluene (3×100 mL) to remove any excess TFA to afford a yellow oil that was used crude.

The crude oil and phthalic anhydride (13.0 g, 93.1 mmol, 3 equiv) were dissolved in anhydrous toluene, after which anhydrous triethylamine (13 mL, 93.3 mmol, 5.5 equiv) was added. The reaction was heated to 100° C. and stirred at reflux overnight. Upon completion, the reaction was cooled and diluted with EtOAc (100 mL). The organic layer was extracted with 1M NaOH (3×100 mL), washed with 20% citric acid (50 mL), aq. sat. NaHCO₃ (50 mL), and brine (50 mL). The organic phase was then dried over Na₂SO₄ and the resulting solution was concentrated and purified by silica gel chromatography (0-50% EtOAc in hexane) to afford 3 as a pale-yellow solid (7.72 g, 92%). ¹H NMR (500 MHz, CDCl₃) δ 7.83 (dd, J=5.4, 3.1 Hz, 2H), 7.72 (dd, J=5.5, 3.0 Hz, 2H), 6.81 (t, J=2.1 Hz, 1H), 5.09 (tt, J=9.6, 7.3 Hz, 1H), 3.76 (s, 3H), 3.11-2.83 (m, 4H). ¹³C NMR (126 MHz, CDCl₃) δ 168.19, 165.05, 141.15, 134.69, 134.19, 132.09, 123.41, 51.70, 48.23, 37.63, 35.83.

### Methyl(1S,3R)-3-(1,3-dioxoisoindolin-2-yl)-6,6-difluorobicyclo[3.1.0]hexane-1-carboxylate (4)

To a 40 mL pressure-release vial, 3 (695.4 mg, 2.56 mmol, 1.0 equiv), NaF (108.1 mg, 2.57 mmol, 1.0 equiv), and anhydrous toluene (20 mL) were added. The solution was then degassed by sparging with nitrogen for 10 min, after which TFDA (3.1 mL, 15.5 mmol, 6.1 equiv) was added dropwise. The reaction mixture was heated to 110° C. overnight, after which time the reaction was allowed to cool to room temperature. The reaction mixture was then diluted with EtOAc (50 mL) and extracted with sat. NaHCO₃ (3×10 mL), washed with 20% citric acid (10 mL), and brine (10 mL). The organic layer was dried over Na₂SO₄, concentrated, and purified by silica gel chromatography (0-50% EtOAc in hexane to provide a mixture of 3 and 4. This mixture was separated via preparative HPLC with a C18 silica gel column (0-55% MeCN in H₂O) to afford 4 as a white solid (402.9 mg, 49%). ¹H NMR (500 MHz, CDCl₃) δ 7.81 (dd, J=5.7, 2.9 Hz, 2H), 7.70 (dd, J=5.5, 3.0 Hz, 2H), 4.83 (tt, J=8.3, 4.2 Hz, 1H), 3.75 (d, J=5.2 Hz, 3H), 2.92 (ddd, J=13.1, 8.4, 4.2 Hz, 1H), 2.78-2.70 (m, 2H), 2.56-2.48 (m, 1H), 2.32 (dd, J=12.5, 8.9 Hz, 1H). ¹³C NMR (126 MHz, CDCl₃) δ 168.08, 167.96, 134.34, 131.90, 123.53, 52.75, 49.40, 49.31, 32.98, 31.03, 29.13. ¹⁹F NMR (376 MHz, CDCl₃) δ-132.81 (dd, J=155.2, 0.6 Hz), −138.63 (d, J=154.8 Hz).

### (1S,3R)-3-amino-6,6-difluorobicyclo[3.1.0]hexane-1-carboxylic acid (6)

To a 100 mL round bottom flask, 4 (375.0 mg, 1.17 mmol, 1.0 equiv) was dissolved in a total of 16 mL DCM: MeOH (2:1), and hydrazine hydrate (0.4 mL, 7.00 mmol, 6.0 equiv, 51% weight) was added dropwise to the mixture. The reaction was allowed to stir overnight, resulting in the formation of white precipitate. The reaction mixture was diluted with DCM (20 mL) and extracted water (2×7 mL). The organic layer was dried on Na₂SO₄, concentrated, and the crude residue was dissolved in 6 M HCl (12 mL), and the reaction was heated to 80° C. under reflux overnight. The mixture was concentrated and purified by C18 silica gel column chromatography (0% MeCN in H₂O) to afford 6 as a white solid (96.1 mg, 39%). ¹H NMR (500 MHz, D₂O) δ 3.78 (qd, J=7.9, 3.1 Hz, 1H), 2.84 (dd, J=13.4, 6.4 Hz, 1H), 2.73 (dd, J=14.3, 8.1 Hz, 1H), 2.58 (dd, J=14.2, 7.9 Hz, 1H), 2.51 (ddd, J=14.2, 7.5, 4.0 Hz, 1H), 2.30 (dtd, J=14.3, 7.1, 3.1 Hz, 1H). ¹³C NMR (126 MHz, D₂O) δ 171.27, 114.36, 50.70, 39.82, 33.00, 32.00, 30.04. 19F NMR (376 MHz, D₂O) δ-132.86 (dd, J=157.3, 13.5 Hz), −139.07 (d, J=157.8 Hz). HR-MS (ESI) calculated [M+H]⁺for C₇H₉F₂NO₂⁺=178.0680, found: 178.0666.

### Synthesis Example 2

The synthesis of 6 is shown in Scheme 3. The synthesis begins with enantiopure Vince lactam (1) which can be Boc protected, followed by ring opening with sodium hydride. The basic ring opening provides a mixture of regioisomers due to epimerization of the alkene to the more favored alpha-beta unsaturated ester (2a). Subjecting the mixture of isomers to DBU at reflux overnight converts them both the more thermodynamically favored unsaturated ester 2a. The Boc group can then be exchanged to a phthalimide via deprotection with TFA and protection of the resulting free amine with phthalic anhydride. The resulting phthalimide 3a can then be subjected to TFDA with catalytic NaF at reflux to yield the desired difluorocyclopropane 4a as a single regioisomer. Deprotection of the phthalimide with hydrazine provides the free amine 5a, which can then be further deprotected with aqueous HCl to the HCl salt of 6.

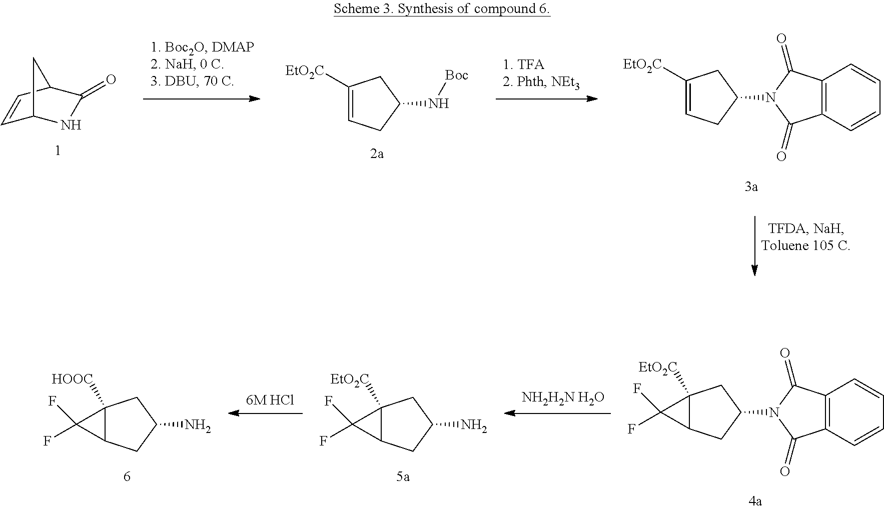

### 1,1-Dimethylethyl(1S,4R)-3-oxo-2-azabicyclo[2.2.1]hept-5-ene-2-carboxylate (2a)

(1S)-(+)-2-azabicyclo[2.2.1]hept-5-en-3-one (4 g, 3.7 mmol, 1 equiv) and DMAP (0.226 g, 0.185 mmol, 0.05 equiv) was dissolved in THF (400 mL), after which Boc anhydride (16 g, 7.4 mmol, 2 equiv) was added portionwise. The resulting solution was allowed to stir at room temperature until consumption of starting material, monitored by LCMS or TLC (70% EtOAc in hexanes). The reaction was directly concentrated under vacuum and azeotroped with water several times to remove excess Boc anhydride. The residue was redissolved in EtOAc (400 mL) and washed with 20% citric acid (2×60 mL), aqueous NaHCO₃ (60 mL), and brine (60 mL). The organic layer was dried with anhydrous Na₂SO₄. The solution was then concentrated down to an orange solid that was used crude in the next reaction.

The crude material was dissolved in anhydrous ethanol (200 mL) and cooled to 0° C., after which NaH (0.1 equiv) was added slowly. The reaction was monitored for consumption of starting material, and a further 0.1 equivalents of NaH was added if the reaction did not proceed within 1 hour. Upon completion, the reaction was quenched with aqueous NH₄Cl (60 mL) and extracted with EtOAc (3×200 mL). The organic layers were combined, washed with brine (60 mL), and dried with anhydrous Na₂SO₄. The organic solution was then dried and used crude in the next step.

The crude mixture of regioisomers was dissolved in anhydrous THF (300 mL), to which was added DBU (1.2 equiv). The resulting solution was heated to reflux overnight then concentrated and directly purified by silica gel chromatography (0-40% EtOAc in hexanes) to afford 2 as a clear oil. Methyl ester analog of compound 2 was previously reported.

### Ethyl(R)-4-(1,3-dioxoisoindolin-2-yl)cyclopent-1-ene-1-carboxylate (3a)

Compound 2 was dissolved in DCM with TFA (0.5-1 mL per gram of 2a) and allowed to stir until consumption of starting material. The solution was concentrated and azeotroped with toluene to remove any excess TFA. The resulting crude TFA salt (4.82 g, 1.79 mmol) was dissolved in anhydrous toluene (200 mL) with phthalic anhydride (7.53 g, 5.91 mmol, 3 equiv) and triethyl amine (2.54 mL, 3.0 mmol, 1.5 equiv) and heated at reflux overnight. The reaction was concentrated, washed with 1 M NaOH (2×40 mL), 20% citric acid (60 mL), aqueous NaHCO₃ (60 mL), and brine (60 mL). The organic layer was dried with anhydrous Na₂SO₄ and concentrated. The resulting residue was purified by silica gel chromatography (0-100% EtOAc in hexanes) to afford 3a as a white solid (yield greater than 1.5 g). 1H NMR (500 MHz, CDCl₃) δ 7.82 (dq, J=6.2, 2.0 Hz, 2H), 7.70 (dp, J=8.1, 2.9 Hz, 2H), 6.78 (t, J=2.4 Hz, 1H), 5.07 (p, J=9.1 Hz, 1H), 4.23-4.15 (m, 2H), 3.09-2.99 (m, 2H), 2.94 (dd, J=16.3, 9.9 Hz, 1H), 2.90-2.77 (m, 1H), 1.28 (tq, J=7.1, 1.3 Hz, 3H). LRMS: [M+H⁺]=286

### Ethyl(1S,3R)-3-(1,3-dioxoisoindolin-2-yl)-6,6-difluorobicyclo[3.1.0]hexane-1-carboxylate (4a)

To a 40 mL vial was added 3a (0.707 g, 2.5 mmol), NaF (50 mg, 1.25 mmol, 0.5 equiv) and dry toluene (20 mL). The solution was degassed by sparging with nitrogen before the dropwise addition of TFDA (2 mL, 9.9 mmol, 4.0 equiv). The reaction was then heated to 105° C. for 1 hour and then diluted with EtOAc (10 mL) and extracted with aqueous NaHCO₃ (2×5 mL), 20% citric acid (2×5 mL), and brine (5 mL). The organic layer was dried with anhydrous Na₂SO₄ and concentrated before the resulting residue was purified by silica gel chromatography (0-50% EtOAc in hexanes) to afford 4a as a white solid (758 mg, 91%). ¹H NMR (500 MHz, CDCl₃) δ 7.83 (q, J=3.8 Hz, 2H), 7.72 (q, J=3.9 Hz, 2H), 4.84 (ddt, J=12.3, 8.2, 4.3 Hz, 1H), 4.23 (pd, J=7.5, 3.6 Hz, 2H), 2.93 (ddt, J=12.8, 7.9, 3.6 Hz, 1H), 2.75 (dq, J=8.5, 5.0 Hz, 2H), 2.53 (dd, J=13.5, 9.6 Hz, 1H), 2.33 (ddt, J=13.8, 9.0, 4.4 Hz, 1H), 1.29 (td, J=7.2, 2.8 Hz, 3H). LRMS: [M+H¹]=386

### (1S,3R)-3-amino-6,6-difluorobicyclo[3.1.0]hexane-1-carboxylic acid HCl salt (6)

To a 40 mL vial was added 4a (0.160 g, 0.478 mmol) and a 2:1 mixture of MeOH and DCM (5 mL total). To this mixture was added hydrazine hydrate (0.143 g, 2.87 mmol, 6 equiv) dropwise. The reaction was allowed to stir overnight and monitored for consumption. The reaction was extracted with DCM (4×5 mL), the organic layers were collected and dried over anhydrous Na₂SO₄ to yield a yellow oil. The oil was suspended in DCM and the salt was formed via addition of 3 M HCl in ether (2 mL). The product was then purified via C18 silica gel chromatography (10-90% MeCN in water) to yield 5a which was directly subjected to 6 M HCl at reflux for 4 hours. The reaction was then concentrated and then purified via C18 silica gel chromatography (0-40% MeCN in water) to yield 6 as a tan solid. 1H NMR (500 MHz, D₂O) δ 3.70 (qt, J=10.5, 4.8 Hz, 1H), 2.74 (dd, J=13.4, 6.4 Hz, 1H), 2.63 (dd, J=14.3, 7.9 Hz, 1H), 2.48 (dd, J=14.2, 7.8 Hz, 1H), 2.41 (ddd, J=14.2, 7.4, 4.0 Hz, 1H), 2.20 (dtd, J=14.3, 7.1, 3.1 Hz, 1H). 19F NMR (376 MHz, D₂O) 8-132.80 (dd, J=157.2, 13.5 Hz), −138.84 (dd, J=156.7, 3.7 Hz). LRMS: [M+H⁺]=178.

### CONCLUSION

Our current work has confirmed that 6 is a mechanism-based inactivator of hOAT, with modest activity in GABA-AT. We have been able to determine that the partition ratio of the inhibitor is 7.5, and that the final adduct is a slow-tight binder that is very tightly bound in the pocket.

### REFERENCES

(1) Madsen, K. K.; Larsson, O. M.; Schousboe, A. Regulation of Excitation by GABA Neurotransmission: Focus on Metabolism and Transport. In Inhibitory Regulation of Excitatory Neurotransmission, Darlison, M. G. Ed.; Springer Berlin Heidelberg, 2008; pp 201-221.
(2) Karlsson, A.; Fonnum, F.; Malthe-Sørenssen, D.; Storm-Mathisen, J. Effect of the convulsive agent 3-mercaptopropionic acid on the levels of GABA, other amino acids and glutamate decarboxylase in different regions of the rat brain. Biochemical Pharmacology 1974, 23 (21), 3053-3061. DOI: https://doi.org/10.1016/0006-2952 (74) 90281-0.
(3) Jeremiah, S.; Povey, S. The biochemical genetics of human γ-aminobutyric acid transaminase. Annals of Human Genetics 1981, 45 (3), 231-236, https://doi.org/10.1111/j.1469-1809.1981.tb00334.x. DOI: https://doi.org/10.1111/j.1469-1809.1981.tb00334.x (accessed 2023 Feb. 1). Durkin, M. M.; Smith, K. E.; Borden, L. A.; Weinshank, R. L.; Branchek, T. A.; Gustafson, E. L. Localization of messenger RNAs encoding three GABA transporters in rat brain: an in situ hybridization study. Molecular Brain Research 1995, 33 (1), 7-21. DOI: https://doi.org/10.1016/0169-328X (95) 00101-W.
(4) Gale, K. GABA in Epilepsy: The Pharmacologic Basis. Epilepsia 1989, 30 (s3), S1-S11, https://doi.org/10.1111/j.1528-1157.1989.tb05825.x. DOI: https://doi.org/10.1111/j.1528-1157.1989.tb05825.x (accessed 2023 Feb. 1).
(5) de Boer, H. M.; Mula, M.; Sander, J. W. The global burden and stigma of epilepsy. Epilepsy & Behavior 2008, 12 (4), 540-546. DOI: https://doi.org/10.1016/j.yebeh.2007.12.019.
(6) Woodbury, D. M.; Penry, J. K.; Pippenger, C. Antiepileptic drugs; Raven Press, 1982.
(7) Bialer, M.; White, H. S. Key factors in the discovery and development of new antiepileptic drugs. Nature Reviews Drug Discovery 2010, 9 (1), 68-82. DOI: 10.1038/nrd2997.
(8) Yogeeswari, P.; Sriram, D.; Vaigundaragavendran, J. The GABA Shunt: An Attractive and Potential Therapeutic Target in the Treatment of Epileptic Disorders. Current Drug Metabolism 2005, 6 (2), 127-139. DOI: http://dx.doi.org/10.2174/1389200053586073.
(9) Iversen, L. L.; Bird, E. D.; Mackay, A. V. P.; Rayner, C. N. Analysis of glutamate decarboxylase in post-mortem brain tissue in Huntington's chorea. Journal of Psychiatric Research 1974, 11, 255-256. DOI: https://doi.org/10.1016/0022-3956 (74) 90099-5.
(10) Nishino, N.; Fujiwara, H.; Noguchi-Kuno, S. A.; Tanaka, C. GABAA Receptor but Not Muscarinic Receptor Density Was Decreased in the Brain of Patients with Parkinson's Disease. Japanese Journal of Pharmacology 1988, 48 (3), 331-339. DOI: https://doi.org/10.1254/jjp.48.331.
(11) Aoyagi, T.; Wada, T.; Nagai, M.; Kojima, F.; Harada, S.; Takeuchi, T.; Takahashi, H.; Hirokawa, K.; Tsumita, T. Increased &gamma; Aminobutyrate Aminotransferase Activity in Brain of Patients with Alzheimer's Disease. CHEMICAL & PHARMACEUTICAL BULLETIN 1990, 38 (6), 1748-1749. DOI: 10.1248/cpb.38.1748.
(12) Takanaga, H.; Ohtsuki, S.; Hosoya, K.-I.; Terasaki, T. GAT2/BGT-1 as a System Responsible for the Transport of γ-Aminobutyric Acid at the Mouse Blood-Brain Barrier. Journal of Cerebral Blood Flow & Metabolism 2001, 21 (10), 1232-1239. DOI: 10.1097/00004647-200110000-00012 (accessed 2023 Feb. 1).
(13) Kakee, A.; Takanaga, H.; Terasaki, T.; Naito, M.; Tsuruo, T.; Sugiyama, Y. Efflux of a suppressive neurotransmitter, GABA, across the blood-brain barrier. Journal of Neurochemistry 110-118, https://doi.org/10.1046/j.1471-4159.2001.00540.x. DOI: 2001, 79 (1), https://doi.org/10.1046/j.1471-4159.2001.00540.x (accessed 2023 Feb. 1).
(14) Silverman, R. B. Mechanism-based enzyme inactivators. In Methods in Enzymology, Vol. 249; Academic Press, 1995; pp 240-283. Silverman, R. B. Mechanism-based Inactivation Enzyme Inactivation: Chemistry and Enzymology; CRC, 1988.
(15) Hwang, B.-Y.; Cho, B.-K.; Yun, H.; Koteshwar, K.; Kim, B.-G. Revisit of aminotransferase in the genomic era and its application to biocatalysis. Journal of Molecular Catalysis B: Enzymatic 2005, 37 (1), 47-55. DOI: https://doi.org/10.1016/j.molcatb.2005.09.004.
(16) Herzfeld, A.; Knox, W. E. The Properties, Developmental Formation, and Estrogen Induction of Ornithine Aminotransferase in Rat Tissues. Journal of Biological Chemistry 1968, 243 (12), 3327-3332. DOI: https://doi.org/10.1016/S0021-9258 (18) 93310-7.
(17) Storici, P.; Capitani, G.; Müller, R.; Schirmer, T.; Jansonius, J. N. Crystal structure of human ornithine aminotransferase complexed with the highly specific and potent inhibitor 5-fluoromethylornithine111Edited by R. Huber. Journal of Molecular Biology 1999, 285 (1), 297-309. DOI: https://doi.org/10.1006/jmbi.1998.2289. Markova, M.; Peneff, C.; Hewlins, M. J. E.; Schirmer, T.; John, R. A. Determinants of Substrate Specificity in ω-Aminotransferases*. Journal of Biological Chemistry 2005, 280 (43), 36409-36416. DOI: https://doi.org/10.1074/jbc.M506977200.
(18) Lee, H.; Juncosa, J. I.; Silverman, R. B. Ornithine Aminotransferase versus GABA Aminotransferase: Implications for the Design of New Anticancer Drugs. Medicinal Research Reviews 2015, 35 (2), 286-305, https://doi.org/10.1002/med.21328. DOI https://doi.org/10.1002/med.21328 (accessed 2023 Feb. 1).
(19) Pan, Y.; Qiu, J.; Silverman, R. B. Design, Synthesis, and Biological Activity of a Difluoro-Substituted, Conformationally Rigid Vigabatrin Analogue as a Potent γ-Aminobutyric Acid Aminotransferase Inhibitor. Journal of Medicinal Chemistry 2003, 46 (25), 5292-5293. DOI: 10.1021/jm034162s.
(20) Silverman, R. B. The 2011 E. B. Hershberg Award for Important Discoveries in Medicinally Active Substances: (1S,3S)-3-Amino-4-difluoromethylenyl-1-cyclopentanoic Acid (CPP-115), a GABA Aminotransferase Inactivator and New Treatment for Drug Addiction and Infantile Spasms. Journal of Medicinal Chemistry 2012, 55 (2), 567-575. DOI: 10.1021/jm201650r.
(21) Moschitto, M. J.; Silverman, R. B. Synthesis of(S)-3-Amino-4-(difluoromethylenyl)-cyclopent-1-ene-1-carboxylic Acid (OV329), a Potent Inactivator of γ-Aminobutyric Acid Aminotransferase. Organic Letters 2018, 20 (15), 4589-4592. DOI: 10.1021/acs.orglett.8b01872.
(22) Shen, S.; Doubleday, P. F.; Weerawarna, P. M.; Zhu, W.; Kelleher, N. L.; Silverman, R. B. Mechanism-Based Design of 3-Amino-4-Halocyclopentenecarboxylic Acids as Inactivators of GABA Aminotransferase. ACS Medicinal Chemistry Letters 2020, 11 (10), 1949-1955. DOI: 10.1021/acsmedchemlett.9b00672.
(23) Silverman, R. B. Design and Mechanism of GABA Aminotransferase Inactivators. Treatments for Epilepsies and Addictions. Chemical Reviews 2018, 118 (7), 4037-4070. DOI: 10.1021/acs.chemrev.8b00009. Nanavati, S. M.; Silverman, R. B. Design of potential anticonvulsant agents: mechanistic classification of GABA aminotransferase inactivators. Journal of Medicinal Chemistry 1989, 32 (11), 2413-2421. DOI: 10.1021/jm00131a001.
(24) Ballone, A.; Lau, R. A.; Zweipfenning, F. P. A.; Ottmann, C. A new soaking procedure for X-ray crystallographic structural determination of protein-peptide complexes. Acta Crystallogr F Struct Biol Commun 2020, 76 (Pt 10), 501-507. DOI: 10.1107/s2053230x2001122x From NLM

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

We claim:

1. A compound of the following formula or a dissociated form, a non-protonated form, a zwitterion form, or a salt thereof: 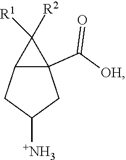
wherein R¹ and R² are independently hydrogen or halo, providing at least one of R¹ and R² is halo.

2. An enantiomerically pure form of the compound of claim 1 having the following formula:

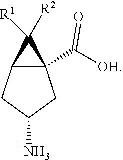

3. The compound of claim 1 or 2, wherein at least one of R¹ and R² is F.

4. The compound of claim 3, wherein R¹ and R² are F.

5. The compound of any one of claims 1-4, wherein the compound is a salt comprising a substituent that is an ammonium substituent or a carboxylate substituent.

6. The compound of claim 5, wherein the ammonium substituent has a counter ion that is a conjugate base of a protic acid.

7. The compound of any one of claims 1-5 in zwitterion form comprising the ammonium substituent and the carboxylate substituent.

8. The compound of claim 1 of a formula:

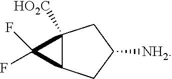

9. A pharmaceutical composition comprising the compound of any one of claims 1-8 and a pharmaceutically suitable carrier, diluent, or excipient.

10. A method of modulating ornithine aminotransferase (OAT) activity, the method comprising contacting the compound of any one of claims 1-8, or the pharmaceutical composition of claim 9, with a medium comprising an OAT, wherein the compound is present in an amount sufficient to modulate OAT activity.

11. The method of claim 10, wherein the contact is in vivo.

12. The method of claim 15 or 16, wherein the OAT is human ornithine aminotransferase (hOAT).

13. A method of reducing activity of an OAT expressed by a human cancer, the method comprising contacting the compound of any one of claims 1-8, or the pharmaceutical composition of claim 9, with the cancer expressing an OAT, wherein the compound is present in an amount that is effective to reduce OAT activity.

14. The method of claim 13, wherein the contact is in vivo.

15. The method of claim 13 or 14, wherein the contact is with a human subject in need of a treatment for cancer.

16. The method of any one of claims 13-15, wherein the OAT is human ornithine aminotransferase (hOAT).

17. A method for treating cancer in a subject in need thereof, the method comprising administering to the subject a therapeutically effective amount of the compound of any one of claims 1-8, or the pharmaceutical composition of claim 9.

18. The method of claim 17, wherein the cancer is characterized by expression or overexpression of human ornithine aminotransferase (hOAT).

19. The method of claim 17 or 18, wherein the cancer is hepatocellular carcinoma (HCC).

20. A method of modulating γ-Aminobutyric acid aminotransferase (GABA-AT) activity, the method comprising contacting the compound of any one of claims 1-8, or the pharmaceutical composition of claim 9, with a medium comprising a GABA-AT, wherein the compound is present in an amount sufficient to modulate GABA-AT activity.

21. The method of claim 20, wherein the contact is in vivo.

22. A method for treating a disease or disorder associated with GABA-AT activity in a subject in need thereof, the method comprising administering to the subject a therapeutically effective amount of the compound of any one of claims 1-8, or the pharmaceutical composition of claim 9.

23. The method of claim 22, wherein the disease or disorder is a neurological disease or disorder.

24. The method of claim 22 or 23, wherein the disease or disorder is epilepsy.
